(19) United States
(12) Patent Application Publication
(10) Pub. No.: US 2007/0253985 A1
(43) Pub. Date: Nov. 1, 2007

(54) NOVEL PROCESSES FOR COATING CONTAINER MEANS WHICH INHIBIT PRECIPITATION OF POLYSACCHARIDE-PROTEIN CONJUGATE FORMULATIONS

(75) Inventors: Jee Loon Look, Gaithersburg, MD (US); Zhaowei Jin, Cary, NC (US); April Longoria, Holly Springs, NC (US); Robert C. Seid, Gaithersburg, MD (US)

Correspondence Address: ALSTON & BIRD LLP, BANK OF AMERICA PLAZA, 101 SOUTH TRYON STREET, SUITE 4000, CHARLOTTE, NC, 28280-4000, US

(73) Assignee: Wyeth, Madison, NJ (US)

(21) Appl. No.: 11/737,697

(22) Filed: Apr. 19, 2007

Related U.S. Application Data
(60) Provisional application No. 60/795,098, filed on Apr. 26, 2006.

(51) Int. Cl. A61K 39/09

(52) U.S. Cl. 424/244.1

(57) ABSTRACT

The present invention relates to processes for preventing particulate formation (e.g., aggregation, precipitation) of polysaccharide-protein conjugates comprised in a container means. In certain embodiments, the invention relates to processes for preventing particulate formation of polysaccharide-protein conjugates which are processed, developed, formulated, manufactured and/or stored in container means such as fermentors, bioreactors, vials, flasks, bags, syringes, rubber stoppers, tubing and the like.

### CROSS REFERENCE TO RELATED APPLICATIONS

This application claims the benefit of U.S. Provisional Application No. 60/795,098, filed Apr. 26, 2006, which is hereby incorporated in its entirety by reference herein.

### FIELD OF THE INVENTION

The present invention generally relates to the fields of immunology, bacteriology, vaccine formulation, protein stability and process development. More particularly, the invention relates to processes for inhibiting aggregation of polysaccharide-protein conjugate formulations comprised in container means.

### BACKGROUND OF THE INVENTION

It is generally accepted in the bio-pharmaceutical arts, that improving the stability of an immunogenic composition (e.g., a polysaccharide-protein conjugate formulation) is a necessary and highly desirable goal. For example, an immunogenic composition must appear fresh, elegant and professional when administered to a patient. Any changes in stability and/or physical appearance of the immunogenic composition, such as color change, clouding or haziness, may cause a patient or consumer to lose confidence in the product. Furthermore, because many immunogenic formulations are dispensed in multiple-dose containers, uniformity of dose content of the active ingredient (e.g., a polysaccharide-protein conjugate) over time must be assured (e.g., a cloudy solution can lead to a non-uniform dosage pattern). Additionally, the immunogenic composition must be active throughout its “expected” shelf life, wherein any breakdown of the immunogenic composition to an inactive or otherwise undesired form (e.g., an aggregate) lowers the total concentration of the product.

Several reports in the literature have suggested that the stability of a particular immunogenic composition (e.g., a polysaccharide-protein conjugate) is at least in part dependent upon the specific carrier protein (Ho et al., 2001; Ho et al., 2002; Bolgiano et al., 2001). For example, stability analysis of meningococcal C (MenC) polysaccharides and Haemophilus influenzae type b (Hib) polysaccharides, conjugated to either a tetanus toxoid (TT) or a CRM₁₉₇ carrier protein, revealed different stability profiles dependent on the carrier protein (Ho et al., 2002). In another study (Ho et al., 2001), MenC-CRM₁₉₇ conjugates from two different manufacturers were analyzed (Ho et al., 2001), wherein the MenC-CRM₁₉₇ conjugates differed in their conjugation chemistry and length of conjugate polysaccharide (both having the same carrier protein, CRM₁₉₇). Data from this study further indicated that factors such as conjugation chemistry (e.g., reductive amination either directly or via a chemical spacer group), number of conjugation sites, polysaccharide chain length, pH, storage buffer, storage temperature(s) and freeze/thaw cycles also influence the stability of an immunogenic composition.

Thus, when developing a formulation for an immunogenic composition, many factors must be considered to ensure a safe, stable, robust and cost effective product. Such considerations include, but are not limited to, chemical stability of the immunogenic composition (e.g., hydrolysis of saccharide, de-polymerization of polysaccharides, proteolysis or fragmentation of proteins), physical/thermal stability of the immunogenic composition (e.g., aggregation, precipitation, adsorption), compatibility of the immunogenic composition with the container/closure system, interactions between immunogenic composition and inactive ingredients (e.g., buffers, salts, excipients, cryoprotectants), the manufacturing process, the dosage form (e.g., lyophilized, liquid), the environmental conditions encountered during shipping, storage and handling (e.g., temperature, humidity, shear forces), and the length of time between manufacture and usage.

It has been suggested in the art, that silicone oil, which induces protein secondary and tertiary conformational changes, might be responsible for the aggregation/precipitation seen in certain protein pharmaceutical preparations (Jones et al., 2005). For example, several reports in the 1980s implicated the release of silicone oil from disposable plastic syringes as the causative agent in the aggregation of human insulin (Chantelau and Berger, 1985; Chantelau et al., 1986; Chantelau, 1989; Bernstein, 1987; Baldwin, 1988; Collier and Dawson, 1985). Chantelau et al. (1986) observed that after three or more withdrawals from a ten-dose preparation of insulin (using a siliconized disposable syringe), the vial would begin clouding due to silicone oil contamination, thereby resulting in aggregation and deactivation of the insulin (Chantelau et al., 1986). Paradoxically, silicone oil is a necessary component of plastic syringes, as it serves to lubricate the rubber plunger and facilitate transfer of the plunger down the syringe barrel (i.e., silicone oil improves the syringeability of the formulation).

Furthermore, the use of silicone oil is not limited to syringes, as it is used as a coating for glass vials to minimize protein adsorption, as a lubricant to prevent conglomeration of rubber stoppers during filing procedures, as a lubricant critical to the processability/machinability of glass and elastomeric closures and as a lubricant to ease needle penetration of vial rubber stoppers. Additionally, the siliconization of syringes, glass vials, rubber stoppers and the like, is not a well controlled nor standardized process, and as such, there is a high degree of variability of the silicone oil content from one lot to another.

Thus, there is an ongoing need in the art to optimize the stability of immunogenic compositions such as polysaccharide-protein conjugate formulations.

### SUMMARY OF THE INVENTION

The present invention broadly relates to processes for preventing particulate formation (e.g., aggregation, precipitation) of polysaccharide-protein conjugates comprised in a container means. In certain embodiments, the invention relates to processes for preventing particulate formation of polysaccharide-protein conjugates in the presence of silicone oil. More specifically, in certain embodiments the invention relates to processes for preventing particulate formation of polysaccharide-protein conjugates which are processed, developed, formulated, manufactured and/or stored in container means such as fermentors, bioreactors, vials, flasks, bags, syringes, rubber stoppers, tubing and the like.

Thus, in certain embodiments, the invention is directed to a process for inhibiting precipitation of a polysaccharide-protein conjugate formulation comprised in a container means, the process comprising coating the container means with a water/surfactant solution and adding a polysaccharide-protein conjugate formulation to the coated container means. In certain embodiments, the container means coated with the water/surfactant solution is dried before adding the polysaccharide-protein conjugate formulation to the container means. In one particular embodiment, the coated container means is dried at 70° C. In yet other embodiments, the coated container means is dried at room temperature. In certain other embodiments, the container means is selected from one or more of the group consisting of a vial, a vial stopper, a vial closure, a glass closure, a rubber closure, a plastic closure, a syringe, a syringe stopper, a syringe plunger, a flask, a beaker, a graduated cylinder, a fermentor, a bioreactor, tubing, a pipe, a bag, a jar, an ampoule, a cartridge and a disposable pen.

In other embodiments, the surfactant is selected from the group consisting of polysorbate 20 (Tween™20), polysorbate 40 (Tween™40), polysorbate 60 (Tween™60), polysorbate 65 (Tween™65), polysorbate 80 (Tween™80), polysorbate 85 (Tween™85), Triton™ N-101, Triton™ X-100, oxtoxynol 40, nonoxynol-9, triethanolamine, triethanolamine polypeptide oleate, polyoxyethylene-660 hydroxystearate (PEG-15, Solutol H15), polyoxyethylene-35-ricinoleate (Cremophor EL™), soy lecithin and a poloxamer. In one particular embodiment, the surfactant is polysorbate 80. In certain other embodiments, the final concentration of the polysorbate 80 in the water/surfactant solution is at least 0.1% to 10% polysorbate 80 by volume of the water/surfactant solution. In another embodiment, the final concentration of the polysorbate 80 in the water/surfactant solution is 0.1% polysorbate 80 by volume of the water/surfactant solution. In still other embodiments, the water in the water/surfactant solution is further defined as Water For Injection (WFI).

In certain other embodiments, the polysaccharide-protein conjugate formulation comprises one or more pneumococcal polysaccharides. In one particular embodiment, the one or more pneumococcal polysaccharides are a S. pneumoniae serotype 4 polysaccharide, a S. pneumoniae serotype 6B polysaccharide, a S. pneumoniae serotype 9V polysaccharide, a S. pneumoniae serotype 14 polysaccharide, a S. pneumoniae serotype 18C polysaccharide, a S. pneumoniae serotype 19F polysaccharide, a S. pneumoniae serotype 23F polysaccharide, a S. pneumoniae serotype 1 polysaccharide, a S. pneumoniae serotype 3 polysaccharide, a S. pneumoniae serotype 5 polysaccharide, a S. pneumoniae serotype 6A polysaccharide, a S. pneumoniae serotype 7F polysaccharide and a S. pneumoniae serotype 19A polysaccharide. In other embodiments, the polysaccharide-protein conjugate formulation further comprises one or more meningococcal polysaccharides and/or one or more streptococcal polysaccharides.

In another embodiment, the protein of the polysaccharide-protein conjugate formulation is selected from the group consisting of CRM₁₉₇, a tetanus toxoid, a cholera toxoid, a pertussis toxoid, an E. coli heat labile toxoid (LT), a pneumolysin toxoid, pneumococcal surface protein A (PspA), pneumococcal adhesin protein A (PsaA), a C5a peptidase from Streptococcus, Haemophilus influenzae protein D, ovalbumin, keyhole limpet haemocyanin (KLH), bovine serum albumin (BSA) and purified protein derivative of tuberculin (PPD).

In one particular embodiment, the polysaccharide-protein conjugate formulation is a 7-valent pneumococcal conjugate (7vPnC) formulation comprising a S. pneumoniae serotype 4 polysaccharide conjugated to a CRM₁₉₇ polypeptide, a S. pneumoniae serotype 6B polysaccharide conjugated to a CRM₁₉₇ polypeptide, a S. pneumoniae serotype 9V polysaccharide conjugated to a CRM₁₉₇ polypeptide, a S. pneumoniae serotype 14 polysaccharide conjugated to a CRM₁₉₇ polypeptide, a S. pneumoniae serotype 18C polysaccharide conjugated to a CRM₁₉₇ polypeptide, a S. pneumoniae serotype 19F polysaccharide conjugated to a CRM₁₉₇ polypeptide and a S. pneumoniae serotype 23F polysaccharide conjugated to a CRM₁₉₇ polypeptide.

In other embodiments, the polysaccharide-protein conjugate formulation is a 13-valent pneumococcal conjugate (13vPnC) formulation comprising a S. pneumoniae serotype 4 polysaccharide conjugated to a CRM₁₉₇ polypeptide, a S. pneumoniae serotype 6B polysaccharide conjugated to a CRM₁₉₇ polypeptide, a S. pneumoniae serotype 9V polysaccharide conjugated to a CRM₁₉₇ polypeptide, a S. pneumoniae serotype 14 polysaccharide conjugated to a CRM₁₉₇ polypeptide, a S. pneumoniae serotype 18C polysaccharide conjugated to a CRM₁₉₇ polypeptide, a S. pneumoniae serotype 19F polysaccharide conjugated to a CRM₁₉₇ polypeptide, a S. pneumoniae serotype 23F polysaccharide conjugated to a CRM₁₉₇ polypeptide, a S. pneumoniae serotype 1 polysaccharide conjugated to a CRM₁₉₇ polypeptide, a S. pneumoniae serotype 3 polysaccharide conjugated to a CRM₁₉₇ polypeptide, a S. pneumoniae serotype 5 polysaccharide conjugated to a CRM₁₉₇ polypeptide, a S. pneumoniae serotype 6A polysaccharide conjugated to a CRM₁₉₇ polypeptide, a S. pneumoniae serotype 7F polysaccharide conjugated to a CRM₁₉₇ polypeptide and a S. pneumoniae serotype 19A polysaccharide conjugated to a CRM₁₉₇ polypeptide.

In another embodiment, the invention is directed to a process for inhibiting precipitation of a polysaccharide-protein conjugate formulation contained in a container means, the process comprising coating the container means with an ethanol/surfactant solution and adding a polysaccharide-protein conjugate formulation to the coated container means. In certain embodiments, the ethanol/surfactant coated container means is dried before adding the polysaccharide-protein conjugate formulation. In one particular embodiment, the coated container means is dried at 70° C. In yet another embodiment, the coated container means is dried at room temperature.

In certain embodiments, the container means is selected from one or more of the group consisting of a vial, a vial stopper, a vial closure, a glass closure, a rubber closure, a plastic closure, a syringe, a syringe stopper, a syringe plunger, a flask, a beaker, a graduated cylinder, a fermentor, a bioreactor, tubing, a pipe, a bag, a jar, an ampoule, a cartridge and a disposable pen.

In other embodiments, the surfactant is selected from the group consisting of polysorbate 20 (Tween™20), polysorbate 40 (Tween™40), polysorbate 60 (Tween™60), polysorbate 65 (Tween™65), polysorbate 80 (Tween™80), polysorbate 85 (Tween™85), Triton™ N-101, Triton™ X-100, oxtoxynol 40, nonoxynol-9, triethanolamine, triethanolamine polypeptide oleate, polyoxyethylene-660 hydroxystearate (PEG-15, Solutol H15), polyoxyethylene-35-ricinoleate (Cremophor EL™), soy lecithin and a poloxamer. In one particular embodiment, the surfactant is polysorbate 80. In another embodiment, the final concentration of the polysorbate 80 in the ethanol/surfactant solution is at least 0.1% to 10% polysorbate 80 by volume of the ethanol/surfactant solution. In certain other embodiments, the final concentration of the polysorbate 80 in the ethanol/surfactant solution is 0.1% polysorbate 80 by volume of the ethanol/surfactant solution. In still other embodiments, the ethanol in the ethanol/surfactant solution is 190 proof ethanol.

In another embodiment, the polysaccharide-protein conjugate formulation comprises one or more pneumococcal polysaccharides. In certain embodiments, the one or more pneumococcal polysaccharides are a S. pneumoniae serotype 4 polysaccharide, a S. pneumoniae serotype 6B polysaccharide, a S. pneumoniae serotype 9V polysaccharide, a S. pneumoniae serotype 14 polysaccharide, a S. pneumoniae serotype 18C polysaccharide, a S. pneumoniae serotype 19F polysaccharide, a S. pneumoniae serotype 23F polysaccharide, a S. pneumoniae serotype 1 polysaccharide, a S. pneumoniae serotype 3 polysaccharide, a S. pneumoniae serotype 5 polysaccharide, a S. pneumoniae serotype 6A polysaccharide, a S. pneumoniae serotype 7F polysaccharide and a S. pneumoniae serotype 19A polysaccharide. In certain other embodiments, the polysaccharide-protein conjugate formulation further comprises one or more meningococcal polysaccharides and/or one or more streptococcal polysaccharides.

In yet other embodiments, the protein of the polysaccharide-protein conjugate formulation is selected from the group consisting of CRM₁₉₇, a tetanus toxoid, a cholera toxoid, a pertussis toxoid, an E. coli heat labile toxoid (LT), a pneumolysin toxoid, pneumococcal surface protein A (PspA), pneumococcal adhesin protein A (PsaA), a C5a peptidase from Streptococcus, Haemophilus influenzae protein D, ovalbumin, keyhole limpet haemocyanin (KLH), bovine serum albumin (BSA) and purified protein derivative of tuberculin (PPD).

In one particular embodiment, the polysaccharide-protein conjugate formulation is a 7-valent pneumococcal conjugate (7vPnC) formulation comprising a S. pneumoniae serotype 4 polysaccharide conjugated to a CRM₁₉₇ polypeptide, a S. pneumoniae serotype 6B polysaccharide conjugated to a CRM₁₉₇ polypeptide, a S. pneumoniae serotype 9V polysaccharide conjugated to a CRM₁₉₇ polypeptide, a S. pneumoniae serotype 14 polysaccharide conjugated to a CRM₁₉₇ polypeptide, a S. pneumoniae serotype 18C polysaccharide conjugated to a CRM₁₉₇ polypeptide, a S. pneumoniae serotype 19F polysaccharide conjugated to a CRM₁₉₇ polypeptide and a S. pneumoniae serotype 23F polysaccharide conjugated to a CRM₁₉₇ polypeptide.

In another embodiment, the polysaccharide-protein conjugate formulation is a 13-valent pneumococcal conjugate (13vPnC) formulation comprising a S. pneumoniae serotype 4 polysaccharide conjugated to a CRM₁₉₇ polypeptide, a S. pneumoniae serotype 6B polysaccharide conjugated to a CRM₁₉₇ polypeptide, a S. pneumoniae serotype 9V polysaccharide conjugated to a CRM₁₉₇ polypeptide, a S. pneumoniae serotype 14 polysaccharide conjugated to a CRM₁₉₇ polypeptide, a S. pneumoniae serotype 18C polysaccharide conjugated to a CRM₁₉₇ polypeptide, a S. pneumoniae serotype 19F polysaccharide conjugated to a CRM₁₉₇ polypeptide, a S. pneumoniae serotype 23F polysaccharide conjugated to a CRM₁₉₇ polypeptide, a S. pneumoniae serotype 1 polysaccharide conjugated to a CRM₁₉₇ polypeptide, a S. pneumoniae serotype 3 polysaccharide conjugated to a CRM₁₉₇ polypeptide, a S. pneumoniae serotype 5 polysaccharide conjugated to a CRM₁₉₇ polypeptide, a S. pneumoniae serotype 6A polysaccharide conjugated to a CRM₁₉₇ polypeptide, a S. pneumoniae serotype 7F polysaccharide conjugated to a CRM₁₉₇ polypeptide and a S. pneumoniae serotype 19A polysaccharide conjugated to a CRM₁₉₇ polypeptide.

In another embodiment, the invention is directed to a process for siliconizing a container means for containing a polysaccharide-protein conjugate formulation, wherein the process inhibits precipitation of the polysaccharide-protein conjugate formulation comprised in the container means, the process comprising coating the container means with a silicone oil/surfactant solution and adding the polysaccharide-protein conjugate formulation to the siliconized container means.

In certain embodiments, the silicone oil/surfactant coated container means is dried before adding the polysaccharide-protein conjugate formulation. In one embodiment, the coated container means is dried at 70° C. In another embodiment, the coated container means is dried at room temperature.

In yet another embodiment, the container means is selected from one or more of the group consisting of a vial, a vial stopper, a vial closure, a glass closure, a rubber closure, a plastic closure, a syringe, a syringe stopper, a syringe plunger, a flask, a beaker, a graduated cylinder, a fermentor, a bioreactor, tubing, a pipe, a bag, a jar, an ampoule, a cartridge and a disposable pen.

In other embodiments, the surfactant is selected from the group consisting of polysorbate 20 (Tween™20), polysorbate 40 (Tween™40), polysorbate 60 (Tween™60), polysorbate 65 (Tween™65), polysorbate 80 (Tween™80), polysorbate 85 (Tween™85), Triton™ N-101, Triton™ X-100, oxtoxynol 40, nonoxynol-9, triethanolamine, triethanolamine polypeptide oleate, polyoxyethylene-660 hydroxystearate (PEG-15, Solutol H15), polyoxyethylene-35-ricinoleate (Cremophor EL™), soy lecithin and a poloxamer. In one particular embodiment, the surfactant is polysorbate 80. In another embodiment, the final concentration of the polysorbate 80 in the silicone oil/surfactant solution is at least 0.1% to 10% polysorbate 80 by volume of the silicone oil/surfactant solution. In another embodiment, the final concentration of the polysorbate 80 in the silicone oil/surfactant solution is 0.1% polysorbate 80 by volume of the silicone oil/surfactant solution.

In certain other embodiments, the polysaccharide-protein conjugate formulation comprises one or more pneumococcal polysaccharides. In one particular embodiment, the one or more pneumococcal polysaccharides are a S. pneumoniae serotype 4 polysaccharide, a S. pneumoniae serotype 6B polysaccharide, a S. pneumoniae serotype 9V polysaccharide, a S. pneumoniae serotype 14 polysaccharide, a S. pneumoniae serotype 18C polysaccharide, a S. pneumoniae serotype 19F polysaccharide, a S. pneumoniae serotype 23F polysaccharide, a S. pneumoniae serotype 1 polysaccharide, a S. pneumoniae serotype 3 polysaccharide, a S. pneumoniae serotype 5 polysaccharide, a S. pneumoniae serotype 6A polysaccharide, a S. pneumoniae serotype 7F polysaccharide and a S. pneumoniae serotype 19A polysaccharide. In other embodiments, the polysaccharide-protein conjugate formulation further comprises one or more meningococcal polysaccharides and/or one or more streptococcal polysaccharides.

In another embodiment, the protein of the polysaccharide-protein conjugate formulation is selected from the group consisting of CRM₁₉₇, a tetanus toxoid, a cholera toxoid, a pertussis toxoid, an E. coli heat labile toxoid (LT), a pneumolysin toxoid, pneumococcal surface protein A (PspA), pneumococcal adhesin protein A (PsaA), a C5a peptidase from Streptococcus, Haemophilus influenzae protein D, ovalbumin, keyhole limpet haemocyanin (KLH), bovine serum albumin (BSA) and purified protein derivative of tuberculin (PPD).

In one particular embodiment, the polysaccharide-protein conjugate formulation is a 7-valent pneumococcal conjugate (7vPnC) formulation comprising a S. pneumoniae serotype 4 polysaccharide conjugated to a CRM₁₉₇ polypeptide, a S. pneumoniae serotype 6B polysaccharide conjugated to a CRM₁₉₇ polypeptide, a S. pneumoniae serotype 9V polysaccharide conjugated to a CRM₁₉₇ polypeptide, a S. pneumoniae serotype 14 polysaccharide conjugated to a CRM₁₉₇ polypeptide, a S. pneumoniae serotype 18C polysaccharide conjugated to a CRM₁₉₇ polypeptide, a S. pneumoniae serotype 19F polysaccharide conjugated to a CRM₁₉₇ polypeptide and a S. pneumoniae serotype 23F polysaccharide conjugated to a CRM₁₉₇ polypeptide.

In another embodiment, the polysaccharide-protein conjugate formulation is a 13-valent pneumococcal conjugate (13vPnC) formulation comprising a S. pneumoniae serotype 4 polysaccharide conjugated to a CRM₁₉₇ polypeptide, a S. pneumoniae serotype 6B polysaccharide conjugated to a CRM₁₉₇ polypeptide, a S. pneumoniae serotype 9V polysaccharide conjugated to a CRM₁₉₇ polypeptide, a S. pneumoniae serotype 14 polysaccharide conjugated to a CRM₁₉₇ polypeptide, a S. pneumoniae serotype 18C polysaccharide conjugated to a CRM₁₉₇ polypeptide, a S. pneumoniae serotype 19F polysaccharide conjugated to a CRM₁₉₇ polypeptide, a S. pneumoniae serotype 23F polysaccharide conjugated to a CRM₁₉₇ polypeptide, a S. pneumoniae serotype 1 polysaccharide conjugated to a CRM₁₉₇ polypeptide, a S. pneumoniae serotype 3 polysaccharide conjugated to a CRM₁₉₇ polypeptide, a S. pneumoniae serotype 5 polysaccharide conjugated to a CRM₁₉₇ polypeptide, a S. pneumoniae serotype 6A polysaccharide conjugated to a CRM₁₉₇ polypeptide, a S. pneumoniae serotype 7F polysaccharide conjugated to a CRM₁₉₇ polypeptide and a S. pneumoniae serotype 19A polysaccharide conjugated to a CRM₁₉₇ polypeptide.

In another embodiment, the invention is directed to a polysaccharide-protein conjugate formulation comprised in a container means prepared according to the process of coating a siliconized container means with a water/surfactant solution and adding the polysaccharide-protein conjugate formulation to the coated container means. In certain embodiments, the polysaccharide-protein conjugate formulation is a 7-valent pneumococcal conjugate (7vPnC) formulation. In other embodiments, the polysaccharide-protein conjugate formulation is a 13-valent pneumococcal conjugate (13vPnC) formulation.

In another embodiment, the invention is directed to a polysaccharide-protein conjugate formulation comprised in a container means prepared according to the process of coating a siliconized container means with a water/surfactant solution and adding the polysaccharide-protein conjugate formulation to the coated container means. In one particular embodiment, the polysaccharide-protein conjugate formulation is a 7-valent pneumococcal conjugate (7vPnC) formulation. In yet another embodiment, the polysaccharide-protein conjugate formulation is a 13-valent pneumococcal conjugate (13vPnC) formulation.

In certain other embodiments, the invention is directed to a polysaccharide-protein conjugate formulation comprised in a container means prepared according to the process of coating a container means with a silicone oil/surfactant solution and adding the polysaccharide-protein conjugate formulation to the siliconized container means. In certain embodiments, the polysaccharide-protein conjugate formulation is a 7-valent pneumococcal conjugate (7vPnC) formulation. In certain other embodiments, the polysaccharide-protein conjugate formulation is a 13-valent pneumococcal conjugate (13vPnC) formulation.

Other features and advantages of the invention will be apparent from the following detailed description, from the preferred embodiments thereof, and from the claims.

### DETAILED DESCRIPTION OF THE INVENTION

The present invention addresses an ongoing need in the art to improve the stability of immunogenic compositions such as polysaccharide-protein conjugate formulations. More particularly, the invention described hereinafter, addresses a need in the art for processes that prevent particulate formation (e.g., aggregation, precipitation) of polysaccharide-protein conjugates comprised in container means. As set forth above in the Background of the Invention, silicone oil is often used as (a) a coating for glass vials to minimize protein adsorption, (b) a lubricant to prevent conglomeration of rubber stoppers during filing procedures, (c) a lubricant to ease needle penetration of vial rubber or Teflon® closures, (d) a lubricant of syringe plungers (i.e., to lubricate the rubber plunger and facilitate transfer of the plunger down the syringe barrel and (e) a lubricant critical to the processability/machinability of glass (e.g., vials, ampoules, syringes, beakers, flasks, etc.), plastic (e.g., disposable syringes, vials, bags), elastomers (e.g., rubber stoppers, tubing), stainless steel (e.g., fermentors, reactors) and the like.

Thus, there are many instances during the development, manufacture and storage of a biologic composition (e.g., a polysaccharide-protein conjugate) in which the biologic composition encounters and potentially interacts with silicone oil. The negative impact of the interaction of biologic compositions with silicone oil (i.e., aggregation and precipitation) was first reported with multiple dosage formulations of human insulin (Chantelau and Berger, 1985; Chantelau et al., 1986; Chantelau, 1989; Bernstein, 1987; Baldwin, 1988; Collier and Dawson, 1985). Similarly, it was observed in the present invention (e.g., see Examples I-III), that exposure or interaction of a pneumococcal polysaccharide-protein conjugate with siliconized closures such as syringe stoppers, syringe plungers, glass vials, rubber stoppers and the like, resulted in highly visible particulate formation (i.e., aggregation and precipitation) of pneumococcal polysaccharide-protein conjugate formulations.

As set forth in detail herein, the present invention relates to the unexpected and surprising results that coating a container means with a surfactant such as Tween™80 prevents the aforementioned particulate formation of pneumococcal polysaccharide-protein conjugate formulations. For example, when a siliconized container means (e.g., a siliconized rubber stopper) was placed in a 40 mL glass vial comprising 10 mL of a 13-valent pneumococcal conjugate formulation (60-70 μg/mL) and gently mixed for four hours at room temperature, the conjugate formulation yielded a highly visible white particulate (Example II). In contrast, when the siliconized container means (i.e., the rubber stopper) was coated with a mixture of Tween™80 and water (or a mixture of Tween™80 and silicone oil), prior to being placed in a vial comprising 10 mL of a 13-valent pneumococcal conjugate formulation (60-70 μg/mL) and gently mixed for four hours at room temperature, the precipitation of the 13-valent pneumococcal conjugate was completely inhibited (Example II). It was also observed in a separate experiment, that coating a siliconized container means with a mixture of Tween™80 and water (Example 11), Tween™80 and ethanol (Example 11) or Tween™80 and silicone oil (data not shown), prevented the precipitation of a 13-valent pneumococcal conjugate formulation stored at 8° C. for twenty-four hours.

Thus, as set forth herein, the surfactant coatings of invention stabilize polysaccharide-protein conjugate formulations, comprised in container means, against silicone oil interactions, shear forces, shipping agitation and the like. The invention described hereinafter is therefore directed to processes that prevent particulate formation (e.g., aggregation, precipitation) of polysaccharide-protein conjugates comprised in a container means.

In one particular embodiment, the invention is directed to a process for inhibiting precipitation of a polysaccharide-protein conjugate formulation comprised in a container means, the process comprising coating the container means with a water/surfactant solution and adding a polysaccharide-protein conjugate formulation to the coated container means. In another embodiment, the invention is directed to a process for inhibiting precipitation of a polysaccharide-protein conjugate formulation comprised in a container means, the process comprising coating the container means with an ethanol/surfactant solution and adding a polysaccharide-protein conjugate formulation to the coated container means. In still another embodiment, the invention is directed to a process for siliconizing a container means for containing a polysaccharide-protein conjugate formulation, wherein the process inhibits precipitation of the polysaccharide-protein conjugate formulation comprised in the container means, the process comprising coating the container means with a silicone oil/surfactant solution and adding the polysaccharide-protein conjugate formulation to the siliconized container means.

As defined hereinafter, the terms “precipitation”, “precipitate” “particulate formation”, “clouding” and “aggregation” may be used interchangeably and are meant to refer to any physical interaction or chemical reaction that results in the “aggregation” of a polysaccharide-protein conjugate. The process of aggregation (e.g., protein aggregation) is well known and described in the art, and is often influenced by numerous physicochemical stresses, including heat, pressure, pH, agitation, freeze-thawing, dehydration, heavy metals, phenolic compounds, denaturants and the like.

As defined hereinafter, a “polysaccharide-protein conjugate” of the invention includes liquid, frozen liquid and solid (e.g., freeze-died or lyophilized) polysaccharide-protein conjugate formulations.

As defined hereinafter, a “water/surfactant solution”, a “water/surfactant mixture”, an “ethanol/surfactant solution”, an “ethanol/surfactant mixture”, a “silicone oil/surfactant solution” and a “silicone oil/surfactant mixture” are collectively referred to as “surfactant coatings”, “surfactant mixtures” or “surfactant solutions”.

The novel container means coating processes comprising the surfactant mixtures described above (i.e., ethanol/surfactant, water/surfactant or silicone oil/surfactant), in addition to preventing precipitation of polysaccharide-protein conjugates in the presence of silicone oil, provide several additional advantages/benefits. For example, by using the novel surfactant coatings of the present invention, there is no need to re-formulate a given polysaccharide-protein conjugate formulation to circumvent or reduce precipitation induced via siliconized container means. Additionally, the surfactant coatings are compatible with current siliconized container means such as syringes, syringe stoppers, vials, etc., and as such, there is no need to switch container means manufacturer and/or alter current polysaccharide-protein conjugate processes and manufacturing protocols in order to prevent polysaccharide-protein conjugate precipitation.

### A. CONTAINER MEANS

As set forth above, the present invention is directed to coating processes that prevent particulate formation (e.g., aggregation, precipitation) of polysaccharide-protein conjugates in the presence of silicone oil. In specific embodiments, the coating process comprises coating a siliconized container means with a water/surfactant mixture, an ethanol/surfactant mixture or a silicone oil/surfactant mixture (i.e., a surfactant coating). In another specific embodiment, the coating process is directed to siliconizing a container means with a silicone oil/surfactant mixture. In these specific embodiments, the container means (coated with the silicone oil/surfactant mixture) retains the lubricious benefits of the silicone oil (e.g., a silicone coated syringe plunger) while the surfactant concomitantly inhibits the particulate formation of a polysaccharide-protein conjugate contained in the newly siliconized container means.

As defined herein, a “container means” of the present invention includes any composition of matter which is used to “contain”, “hold”, “mix”, “blend”, “dispense”, “inject”, “transfer”, “nebulize”, etc. a polysaccharide-protein conjugate during research, processing, development, formulation, manufacture, storage and/or administration. For example, a container means of the present invention includes, but is not limited to, general laboratory glassware, flasks, beakers, graduated cylinders, fermentors, bioreactors, tubings, pipes, bags, jars, vials, vial closures (e.g., a rubber stopper, a screw on cap), ampoules, syringes, syringe stoppers, syringe plungers, rubber closures, plastic closures, glass closures, and the like. A container means of the present invention is not limited by material of manufacture, and includes materials such as glass, metals (e.g., steel, stainless steel, aluminum, etc.) and polymers (e.g., thermoplastics, elastomers, thermoplastic-elastomers).

The skilled artisan will appreciate that the container means set forth above are by no means an exhaustive list, but merely serve as guidance to the artisan with respect to the variety of container means which will benefit from surfactant coatings of the present invention. Additional container means contemplated for use in the present invention may be found in published catalogues from laboratory equipment vendors and manufacturers such as United States Plastic Corp. (Lima, Ohio), VWR™ (West Chester, Pa.), BD Biosciences (Franklin Lakes, N.J.), Fisher Scientific International Inc. (Hampton, N.H.) and Sigma-Aldrich (St. Louis, Mo.).

### B. SURFACTANTS

In certain embodiments, a surfactant coating of the invention comprises a water/surfactant solution or mixture. In other embodiments, a surfactant coating of the invention comprises an ethanol/surfactant mixture or solution. In yet other embodiments, a surfactant coating of the invention comprises a silicone oil/surfactant solution or mixture.

A surfactant (or a surface-active agent) is generally defined as (a) a molecule or compound comprising a hydrophilic group or moiety and a lipophilic (hydrophobic) group or moiety and/or (b) a molecule, substance or compound that lowers or reduces surface tension of a solution. As defined herein, a “surfactant” of the present invention is any molecule or compound that lowers the surface tension of a polysaccharide-protein conjugate formulation.

As set forth below (e.g., see Examples I-III), the surfactant used in the experiments described herein was polysorbate 80 (Tween™80). However, a surfactant coating of the invention is not limited to any one surfactant, and as such, a surfactant of the invention comprises any surfactant or any combination of surfactants which stabilize a polysaccharide-protein conjugate formulation against aggregation. Additional surfactants contemplated for use in the present invention include, but are not limited to, polysorbate 20 (Tween™20), polysorbate 40 (Tween™40), polysorbate 60 (Tween™60), polysorbate 65 (Tween™65), polysorbate 85 (Tween™85), Triton™ N-101, Triton™ X-100, oxtoxynol 40, nonoxynol-9, triethanolamine, triethanolamine polypeptide oleate, polyoxyethylene-660 hydroxystearate (PEG-15, Solutol H15), polyoxyethylene-35-ricinoleate (Cremophor EL™), soy lecithin, poloxamer, hexadecylamine, octadecylamine, octadecyl amino acid esters, lysolecithin, dimethyl-dioctadecylammonium bromide, methoxyhexadecylgylcerol, pluronic polyols, polyamines (e.g., pyran, dextransulfate, poly IC, carbopol), peptides (e.g., muramyl peptide and dipeptide, dimethylglycine, tuftsin), oil emulsions, mineral gels (e.g., aluminum phosphate) and immune stimulating complexes (ISCOMS).

A person of skill in the art may readily determine a suitable surfactant or surfactant combination by measuring the surface tension of a particular polysaccharide-protein conjugate formulation in the presence and absence of the surfactant(s). Alternatively, a surfactant is evaluated qualitatively (e.g., visual inspection of particulate formation) or quantitatively (e.g., light scattering, sedimentation velocity centrifugation, optical density) for its ability to reduce, inhibit or prevent polysaccharide-protein conjugate aggregation.

### C. ADJUVANTS AND PHARMACEUTICAL CARRIERS/EXCIPIENTS

The present invention is directed to surfactant coating processes that prevent aggregation of polysaccharide-protein conjugates comprised in container means. In certain embodiments of the invention, a polysaccharide-protein conjugate comprised in a surfactant coated container means further comprises an adjuvant. An adjuvant is a substance that enhances the immune response when administered together with an immunogen or antigen. A number of cytokines or lymphokines have been shown to have immune modulating activity, and thus may be used as adjuvants, including, but not limited to, the interleukins 1-α, 1-β, 2, 4, 5, 6, 7, 8, 10, 12 (see, e.g., U.S. Pat. No. 5,723,127), 13, 14, 15, 16, 17 and 18 (and its mutant forms), the interferons-α, β and γ, granulocyte-macrophage colony stimulating factor (GMCSF, see, e.g., U.S. Pat. No. 5,078,996 and ATCC Accession Number 39900), macrophage colony stimulating factor (MCSF), granulocyte colony stimulating factor (GCSF), and the tumor necrosis factors α and β (TNF). Still other adjuvants useful in this invention include chemokines, including without limitation, MCP-1, MIP-1α, MIP-1β, and RANTES.

In certain embodiments, an adjuvant used to enhance an immune response of a polysaccharide-protein conjugate formulation include, without limitation, MPL™ (3-O-deacylated monophosphoryl lipid A; Corixa, Hamilton, Mont.), which is described in U.S. Pat. No. 4,912,094, which is hereby incorporated by reference. Also suitable for use as adjuvants are synthetic lipid A analogs or aminoalkyl glucosamine phosphate compounds (AGP), or derivatives or analogs thereof, which are available from Corixa (Hamilton, Mont.), and which are described in U.S. Pat. No. 6,113,918, which is hereby incorporated by reference. One such AGP is 2-[(R)-3-Tetradecanoyloxytetradecanoylamino]ethyl 2-Deoxy-4-O-phosphono-3-O-[(R)-3-tetradecanoyoxytetradecanoyl]-2-[(R)-3-tetradecanoyloxytetradecanoyl-amino]-b-D-glucopyranoside, which is also known as 529 (formerly known as RC529). This 529 adjuvant is formulated as an aqueous form or as a stable emulsion (RC529-SE).

Still other adjuvants include mineral oil and water emulsions, aluminum salts (alum), such as aluminum hydroxide, aluminum phosphate, aluminum sulfate etc., Amphigen, Avridine, L121/squalene, D-lactide-polylactide/glycoside, pluronic polyols, muramyl dipeptide, killed Bordetella, saponins, such as Stimulon™ QS-21 (Antigenics, Framingham, Mass.), described in U.S. Pat. No. 5,057,540, which is hereby incorporated by reference, and particles generated therefrom such as ISCOMS (immunostimulating complexes), ISCOMATRIX (CSL Limited, Parkville, Australia), described in U.S. Pat. No. 5,254,339, Mycobacterium tuberculosis, bacterial lipopolysaccharides, synthetic polynucleotides such as oligonucleotides containing a CpG motif (U.S. Pat. No. 6,207,646, which is hereby incorporated by reference), IC-31 (Intercell AG, Vienna, Austria), described in European Patent Nos. 1,296,713 and 1,326,634, a pertussis toxin (PT), or an E. coli heat-labile toxin (LT), particularly LT-K63, LT-R72, PT-K9/G129; see, e.g., International Patent Publication Nos. WO 93/13302 and WO 92/19265, incorporated herein by reference.

Also useful as adjuvants (and carrier proteins) are cholera toxins and mutants thereof, including those described in published International Patent Application number WO 00/18434 (wherein the glutamic acid at amino acid position 29 is replaced by another amino acid (other than aspartic acid), preferably a histidine). Similar CT toxins or mutants are described in published International Patent Application number WO 02/098368 (wherein the isoleucine at amino acid position 16 is replaced by another amino acid, either alone or in combination with the replacement of the serine at amino acid position 68 by another amino acid; and/or wherein the valine at amino acid position 72 is replaced by another amino acid). Other CT toxins are described in published International Patent Application number WO 02/098369 (wherein the arginine at amino acid position 25 is replaced by another amino acid; and/or an amino acid is inserted at amino acid position 49; and/or two amino acids are inserted at amino acid positions 35 and 36).

In certain embodiments, the polysaccharide-protein conjugate formulations of the invention comprise a pharmaceutically acceptable diluent, excipient or a pharmaceutically acceptable carrier. In one embodiment, the pharmaceutically acceptable diluent is sterile water, water for injection, sterile isotonic saline or a biological buffer. The polysaccharide-protein conjugates are mixed with such diluents or carriers in a conventional manner. As used herein the language “pharmaceutically acceptable carrier” is intended to include any and all solvents, dispersion media, coatings, antibacterial and antifungal agents, isotonic and absorption delaying agents, and the like, compatible with administration to humans or other vertebrate hosts. The appropriate carrier is evident to those skilled in the art and will depend in large part upon the route of administration.

For example, excipients that may be present in a polysaccharide-protein conjugate formulation of the invention are preservatives, chemical stabilizers and suspending or dispersing agents. Typically, stabilizers, preservatives and the like are optimized to determine the best formulation for efficacy in the targeted recipient (e.g., a human subject). Examples of preservatives include chlorobutanol, potassium sorbate, sorbic acid, sulfur dioxide, propyl gallate, the parabens, ethyl vanillin, glycerin, phenol, and parachlorophenol. Examples of stabilizing ingredients include casamino acids, sucrose, gelatin, phenol red, N-Z amine, monopotassium diphosphate, lactose, lactalbumin hydrolysate, and dried milk.

In certain embodiments, a polysaccharide-protein conjugate formulation of the invention is prepared for administration to human subjects in the form of, for example, liquids, powders, aerosols, tablets, capsules, enteric-coated tablets or capsules, or suppositories. Thus, the polysaccharide-protein conjugate formulations may also include, but are not limited to, suspensions, solutions, emulsions in oily or aqueous vehicles, pastes, and implantable sustained-release or biodegradable formulations.

The immunogenic compositions of the present invention, are not limited by the selection of the conventional, physiologically acceptable carriers, diluents and excipients such as solvents, buffers, adjuvants, or other ingredients useful in pharmaceutical preparations of the types described above. The preparation of these pharmaceutically acceptable compositions, from the above-described components, having appropriate pH isotonicity, stability and other conventional characteristics is within the skill of the art.

### D. POLYSACCHARIDE-PROTEIN CONJUGATES

As set forth above, the present invention is directed to surfactant coating processes that prevent particulate formation of polysaccharide-protein conjugates comprised in container means. In certain embodiments, a polysaccharide-protein conjugate formulation of the invention comprises one or more pneumococcal polysaccharides. In other embodiments, a polysaccharide-protein conjugate formulation of the invention comprises one or more streptococcal polysaccharides. In yet other embodiments, a polysaccharide-protein conjugate formulation of the invention comprises one or more meningococcal polysaccharides. In still other embodiments, a polysaccharide-protein conjugate formulation of the invention comprises a combination of one or more pneumococcal polysaccharides, one or more streptococcal and/or one or more meningococcal polysaccharides.

As defined hereinafter, the term “polysaccharide” is meant to include any antigenic saccharide element (or antigenic unit) commonly used in the immunologic and bacterial vaccine arts, including, but not limited to, a “saccharide”, an “oligosaccharide”, a “polysaccharide”, a “liposaccharide”, a “lipo-oligosaccharide (LOS)”, a “lipopolysaccharide (LPS)”, a “glycosylate”, a “glycoconjugate” and the like.

In one particular embodiment of the invention, the one or more pneumococcal polysaccharides are a S. pneumoniae serotype 4 polysaccharide, a S. pneumoniae serotype 6B polysaccharide, a S. pneumoniae serotype 9V polysaccharide, a S. pneumoniae serotype 14 polysaccharide, a S. pneumoniae serotype 18C polysaccharide, a S. pneumoniae serotype 19F polysaccharide, a S. pneumoniae serotype 23F polysaccharide, a S. pneumoniae serotype 1 polysaccharide, a S. pneumoniae serotype 3 polysaccharide, a S. pneumoniae serotype 5 polysaccharide, a S. pneumoniae serotype 6A polysaccharide, a S. pneumoniae serotype 7F polysaccharide and a S. pneumoniae serotype 19A polysaccharide.

In certain embodiments, a polysaccharide-protein conjugate formulation is a 7-valent pneumococcal conjugate (7vPnC) formulation comprising a S. pneumoniae serotype 4 polysaccharide conjugated to a CRM₁₉₇ polypeptide, a S. pneumoniae serotype 6B polysaccharide conjugated to a CRM₁₉₇ polypeptide, a S. pneumoniae serotype 9V polysaccharide conjugated to a CRM₁₉₇ polypeptide, a S. pneumoniae serotype 14 polysaccharide conjugated to a CRM₁₉₇ polypeptide, a S. pneumoniae serotype 18C polysaccharide conjugated to a CRM₁₉₇ polypeptide, a S. pneumoniae serotype 19F polysaccharide conjugated to a CRM₁₉₇ polypeptide and a S. pneumoniae serotype 23F polysaccharide conjugated to a CRM₁₉₇ polypeptide.

In certain other embodiments, a polysaccharide-protein conjugate formulation is a 13-valent pneumococcal conjugate (13vPnC) formulation comprising a S. pneumoniae serotype 4 polysaccharide conjugated to a CRM₁₉₇ polypeptide, a S. pneumoniae serotype 6B polysaccharide conjugated to a CRM₁₉₇ polypeptide, a S. pneumoniae serotype 9V polysaccharide conjugated to a CRM₁₉₇ polypeptide, a S. pneumoniae serotype 14 polysaccharide conjugated to a CRM₁₉₇ polypeptide, a S. pneumoniae serotype 18C polysaccharide conjugated to a CRM₁₉₇ polypeptide, a S. pneumoniae serotype 19F polysaccharide conjugated to a CRM₁₉₇ polypeptide, a S. pneumoniae serotype 23F polysaccharide conjugated to a CRM₁₉₇ polypeptide, a S. pneumoniae serotype 1 polysaccharide conjugated to a CRM₁₉₇ polypeptide, a S. pneumoniae serotype 3 polysaccharide conjugated to a CRM₁₉₇ polypeptide, a S. pneumoniae serotype 5 polysaccharide conjugated to a CRM₁₉₇ polypeptide, a S. pneumoniae serotype 6A polysaccharide conjugated to a CRM₁₉₇ polypeptide, a S. pneumoniae serotype 7F polysaccharide conjugated to a CRM₁₉₇ polypeptide and a S. pneumoniae serotype 19A polysaccharide conjugated to a CRM₁₉₇ polypeptide

Polysaccharides are prepared by standard techniques known to those skilled in the art. For example, the capsular polysaccharides set forth in the present invention are prepared from serotypes 1, 3, 4, 5, 6A, 6B, 7F, 9V, 14, 18C, 19A, 19F and 23F of Streptococcus pneumoniae, wherein each serotype is grown in a soy-based medium and the individual polysaccharides are then purified through centrifugation, precipitation, ultra-filtration, and column chromatography. Similarly, streptococcal polysaccharides (e.g., one or more polysaccharides (or oligosaccharides) from a β-hemolytic Streptococcus such as group A Streptococcus, group B Streptococcus, group C Streptococcus and group G Streptococcus) and meningococcal saccharides (e.g., an N. meningitidis lipo-oligosaccharide (LOS) or lipo-polysaccharide (LPS)) are prepared from clinically relevant serotypes or serogroups, using general techniques and methods known to one of skill in the art. The purified polysaccharides are then chemically activated (e.g., via reductive amination) to make the saccharides capable of reacting with the carrier protein. Once activated, each capsular polysaccharide is separately conjugated to a carrier protein (e.g., CRM₁₉₇) to form a glycoconjugate (or alternatively, each capsular polysaccharide is conjugated to the same carrier protein) and formulated into a single dosage formulation.

The chemical activation of the polysaccharides and subsequent conjugation to the carrier protein (i.e., a polysaccharide-protein conjugate) are achieved by conventional means. See, for example, U.S. Pat. Nos. 4,673,574 and 4,902,506.

Carrier proteins are preferably proteins that are non-toxic and non-reactogenic and obtainable in sufficient amount and purity. Carrier proteins should be amenable to standard conjugation procedures. In a particular embodiment of the present invention, CRM₁₉₇ is used as the carrier protein.

CRM₁₉₇ (Wyeth, Sanford, N.C.) is a non-toxic variant (i.e., toxoid) of diphtheria toxin isolated from cultures of Corynebacterium diphtheria strain C7 (β197) grown in casamino acids and yeast extract-based medium. CRM₁₉₇ is purified through ultra-filtration, ammonium sulfate precipitation, and ion-exchange chromatography. Alternatively, CRM₁₉₇ is prepared recombinantly in accordance with U.S. Pat. No. 5,614,382, which is hereby incorporated by reference. Other diphtheria toxoids are also suitable for use as carrier proteins.

In other embodiments, a carrier protein of the invention is an enzymatically inactive streptococcal C5a peptidase (SCP) (e.g., one or more of the SCP variants described in U.S. Pat. No. 6,951,653, U.S. Pat. No. 6,355,255 and U.S. Pat. No. 6,270,775).

Other suitable carrier proteins include inactivated bacterial toxins such as tetanus toxoid, pertussis toxoid, cholera toxoid (e.g., CT E29H, described in International Patent Application WO2004/083251), E. coli LT, E. coli ST, and exotoxin A from Pseudomonas aeruginosa. Bacterial outer membrane proteins such as outer membrane complex c (OMPC), porins, transferrin binding proteins, pneumolysis, pneumococcal surface protein A (PspA), pneumococcal adhesin protein (PsaA), or Haemophilus influenzae protein D, can also be used. Other proteins, such as ovalbumin, keyhole limpet haemocyanin (KLH), bovine serum albumin (BSA) or purified protein derivative of tuberculin (PPD) can also be used as carrier proteins.

After conjugation of the capsular polysaccharide to the carrier protein, the polysaccharide-protein conjugates are purified (enriched with respect to the amount of polysaccharide-protein conjugate) by a variety of techniques. These techniques include concentration/diafiltration operations, precipitation/elution, column chromatography, and depth filtration.

After the individual glycoconjugates are purified, they are compounded to formulate the immunogenic composition of the present invention. Formulation of the polysaccharide-protein conjugates of the present invention can be accomplished using art-recognized methods. For instance, the 13 individual pneumococcal conjugates can be formulated with a physiologically acceptable vehicle to prepare the composition. Examples of such vehicles include, but are not limited to, water, buffered saline, polyols (e.g., glycerol, propylene glycol, liquid polyethylene glycol) and dextrose solutions.

All patents and publications cited herein are hereby incorporated by reference.

### E. EXAMPLES

The following examples are carried out using standard techniques, which are well known and routine to those of skill in the art, except where otherwise described in detail.

The following examples are presented for illustrative purposes, and should not be construed in any way as limiting the scope of this invention.

### Example 1

### Materials and Methods

The polysaccharide-protein conjugate used in this example was a thirteen-valent pneumococcal polysaccharide conjugate (13vPnC) comprising capsular polysaccharides from S. pneumoniae serotypes 4, 6B, 9V, 18C, 19F, 14, 23F, 1, 3, 5, 6A, 7F and 19A, each of which was conjugated to CRM₁₉₇. The capsular polysaccharides are prepared by standard techniques known to those skilled in the art. Briefly, each pneumococcal polysaccharide serotype was grown in a soy-based medium, the individual polysaccharides were then purified through centrifugation, precipitation, ultra-filtration, and column chromatography. The purified polysaccharides were chemically activated for conjugation and each polysaccharide was separately conjugated to a CRM₁₉₇ carrier protein to form a glycoconjugate and formulated into a single dosage formulation.

The chemical activation of the polysaccharides and subsequent conjugation to the carrier protein were achieved by conventional means (e.g., see U.S. Pat. Nos. 4,673,574 and 4,902,506). CRM₁₉₇ (Wyeth, Sanford, N.C.) is a non-toxic variant (i.e., toxoid) of diphtheria toxin isolated from cultures of Corynebacterium diphtheria strain C7 (β197) grown in casamino acids and yeast extract-based medium. CRM₁₉₇ was purified through ultra-filtration, ammonium sulfate precipitation, and ion-exchange chromatography.

Silicone oil (360 Medical Fluid, 1000 CST) was purchased from Dow Corning® (Midland, Mich.). Syringes (BD Hypak SCF™) and syringe stoppers (BD Hypak SCF™) were purchased from BD Biosciences (Franklin Lakes, N.J.). Clear borosilicate vials (VWR TraceClean™, 40 mL) with Teflon®-lined closures were purchased from VWR™ (West Chester, Pa.). Polysorbate 80 (Tween™80) was purchased from J. T. Baker (Mallinckrodt Baker, Inc.; Phillipsburg, N.J.). Ninety five percent ethanol (190 proof) was purchased from Sigma-Aldrich.

Serial concentrations of 0%, 0.001%, 0.01%, 0.1%, 1.0% and 10% polysorbate 80 (Tween™80) in 10 mL of water for injection (WFI) are shown in Table 1 and made as follows:
(a) 0% Tween™80: 10 mL of WFI was added to a 40 mL glass vial;
(b) 0.001% Tween™80: 0.1 μL (0.0001 mL) of Tween™80 was added to 10 mL of WFI in a 40 mL glass vial and then mixed by vortexing;
(c) 0.01% Tween™80: 1.0 μL (0.001 mL) of Tween™80 was added to 10 mL of WFI in a 40 mL glass vial and then mixed by vortexing;
(d) 0.1% Tween™80: 10 μL (0.01 mL) of Tween™80 was added to 10 mL of WFI in a 40 mL glass vial and then mixed by vortexing;
(e) 1% Tween™80: 100 μL (0.1 mL) of Tween™80 was added to 10 mL of WFI in a 40 mL glass vial and then mixed by vortexing, and
(f) 10% Tween™80: 1000 μL (1.0 mL) of Tween™80 was added to 10 mL of WFI in a 40 mL glass vial and then mixed by vortexing.

TABLE 1
SURFACTANT/WATER MIXTURES

| | Vial 1 | Vial 2 | Vial 3 | Vial 4 | Vial 5 | Vial 6 |
|---|---|---|---|---|---|---|
| Final | 0% | 0.001% | 0.01% | 0.1% | 1% | 10% |
| [Tween80] | | | | | | |
| in WFI | | | | | | |
| WFI (mL) | 10 | 10 | 10 | 10 | 10 | 9 |
| Tween 80 (mL) | 0.0000 | 0.0001 | 0.001 | 0.01 | 0.1 | 1 |
| Rubber | 10 | 10 | 10 | 10 | 10 | 10 |
| Stoppers | | | | | | |

Serial concentrations of 0%, 0.001%, 0.01%, 0.1%, 1.0% and 10% polysorbate 80 (Tween™80) in 10 mL of silicone oil are shown in Table 2 and made as follows:
(a) 0% Tween™80: 10 mL of silicone oil was added to a 40 mL glass vial;
(b) 0.001% Tween™80: 0.1 μL (0.0001 mL) of Tween™80 was added to 10 mL of silicone oil in a 40 mL glass vial and then mixed by vortexing;
(c) 0.01% Tween™80: 1.0 μL (0.001 mL) of Tween™80 was added to 10 mL of silicone oil in a 40 mL glass vial and then mixed by vortexing;
(d) 0.1% Tween™80: 10 μL (0.01 mL) of Tween™80 was added to 10 mL of silicone oil in a 40 mL glass vial and then mixed by vortexing;
(e) 1% Tween™80: 100 μL (0.1 mL) of Tween™80 was added to 10 mL of silicone oil in a 40 mL glass vial and then mixed by vortexing, and
(f) 10% Tween™80: 1000 μL (1.0 mL) of Tween™80 was added to 10 mL of silicone oil in a 40 mL glass vial and then mixed by vortexing.

TABLE 2
SURFACTANT/SILICONE OIL MIXTURES

| | Vial 1 | Vial 2 | Vial 3 | Vial 4 | Vial 5 | Vial 6 |
|---|---|---|---|---|---|---|
| Final | 0% | 0.001% | 0.01% | 0.1% | 1% | 10% |
| [Tween80] | | | | | | |
| in silicone oil | | | | | | |
| Silicone oil | 10 | 10 | 10 | 10 | 10 | 9 |
| (mL) | | | | | | |
| Tween80 (mL) | 0.0000 | 0.0001 | 0.001 | 0.01 | 0.1 | 1 |
| Rubber | 10 | 10 | 10 | 10 | 10 | 10 |
| Stoppers | | | | | | |

### Example 2

### Coating a Container Means with a Surfactant Solution Inhibits Polysaccharide-Protein Conjugate Precipitation

Rubber stoppers (BD Hypac 4432 grey stoppers) were added to twelve 40 mL borosilicate glass vials (10 stoppers per vial), wherein the stoppers in each of the twelve vials were coated with 100 μL of a Tween™80/silicone oil solution (six vials; Table 1) or 100 μL of Tween™80/water (WFI) solution (six vials; Table 2) at one of the following Tween™80 concentrations: 0%, 0.001%, 0.01%, 0.1%, 1.0% or 10%. The twelve vials were then vortexed for five minutes to thoroughly coat the stoppers with either the Tween™80/silicone oil solution or Tween™80/WFI solution and subsequently dried in a 70° C. oven for twenty minutes or dried under a halogen lamp overnight. Four coated stoppers from each concentration of Tween™80/silicone oil (i.e., six Tween™80 concentrations) and four coated stoppers from each concentration Tween™80/WFI (i.e., six Tween™80 concentrations) were placed into separate 40 mL glass vials containing 10 mL (60-70 μg/mL) of 13vPnC. The glass vials were placed on an orbital shaker (100 cpm) at room temperature for four hours and then inspected for particulate formation. As shown in Table 3, concentrations of 0.1%, 1.0% and 10% Tween™80 (w/v) in the Tween™80/WFI mixture completely inhibited particulate formation of the 13-valent pneumococcal conjugate composition. Similarly, as shown in Table 4, concentrations of 0.1%, 1.0% and 10% Tween™80 (w/v) in the Tween™80/silicone oil mixture completely inhibited particulate formation of the 13-valent pneumococcal conjugate composition.

TABLE 3
13VPNC STABILITY IN THE PRESENCE OF STOPPERS
COATED WITH SURFACTANT/WATER MIXTURES

| | Final [Tween80] in WFI | | | | | |
|---|---|---|---|---|---|---|
| | 0% | 0.001% | 0.01% | 0.1% | 1% | 10% |
| 13vPnC (mL) | 10 | 10 | 10 | 10 | 10 | 10 |
| # of Stoppers | 4 | 4 | 4 | 4 | 4 | 4 |
| Particulates visible | Yes | Yes | Yes | No | No | No |

TABLE 4
13VPNC STABILITY IN THE PRESENCE OF STOPPERS
COATED WITH SURFACTANT/SILICONE OIL MIXTURES

| | Final [Tween80] in Silicone oil | | | | | |
|---|---|---|---|---|---|---|
| | 0% | 0.001% | 0.01% | 0.1% | 1% | 10% |
| 13vPnC (mL) | 10 | 10 | 10 | 10 | 10 | 10 |
| # of Stoppers | 4 | 4 | 4 | 4 | 4 | 4 |
| Particulates visible | Yes | Yes | Yes | No | No | No |

### Example 3

### Twenty-Four Hour Stability Assessment of Polysaccharide-Protein Conjugates in the Presence of Rubber Stoppers

Serial concentrations of 1.0% and 10% Tween™80 in 10 mL of water for injection (WFI) are shown in Table 5 and made as follows:
(a) 1% Tween™80: 100 μL (0.1 mL) of Tween™80 was added to 9.9 mL of WFI in a 40 mL glass vial and then mixed by vortexing, and
(b) 10% Tween™80: 1000 μL (1.0 mL) of Tween™80 was added to 9.0 mL of WFI in a 40 mL glass vial and then mixed by vortexing.

TABLE 5
SURFACTANT/WATER MIXTURES

| | Vial 1 | Vial 2 | Vial 3 |
|---|---|---|---|
| Final [Tween80] | 0% | 1% | 10% |
| in WFI | | | |
| WFI (mL) | 10 | 9.9 | 9 |
| Tween 80 (mL) | 0 | 0.1 | 1 |

Serial concentrations of 1.0% and 10% Tween™80 in 10 mL of ethanol are shown in Table 6 and made as follows:
(a) 1% Tween™80: 100 μL (0.1 mL) of Tween™80 was added to 9.9 mL of ethanol in a 40 mL glass vial and then mixed by vortexing, and
(b) 10% Tween™80: 1000 μL (1.0 mL) of Tween™80 was added to 10 mL of ethanol in a 40 mL glass vial and then mixed by vortexing.

TABLE 6
SURFACTANT/ETHANOL MIXTURES

| | Vial 1 | Vial 2 | Vial 3 |
|---|---|---|---|
| Final [Tween80] | 0% | 1% | 10% |
| in ethanol | | | |
| Ethanol (mL) | 10 | 9.9 | 9 |
| Tween80 (mL) | 0 | 0.1 | 1 |

Rubber stoppers (BD Hypac 4432 grey stoppers) were added to six 40 mL borosilicate glass vials (5 stoppers per vial), wherein the five stoppers in each of the six vials were coated with 100 μL of either 0% Tween80/WFI, 1.0% Tween80/WFI, 10% Tween80/WFI, 0% Tween80/ethanol, 1.0% Tween80/ethanol or 10% Tween80/ethanol. After twenty-four hours, the stoppers were removed from the vials and placed on parafilm to air dry in a biosafety cabinet.

After drying, the five stoppers from each concentration of Tween80/WFI (i.e., 0%, 1.0% and 10%) and Tween80/ethanol (i.e., 0%, 1.0% and 10%) were placed into separate 40 mL glass vials containing 10 mL (60-70 μL) of 13vPnC. The vials were then stored at 8° C. for twenty-four hours and visually inspected for particulate matter. As set forth below in Tables 7 and 8, there was no observable particulate formation of the 13-valent pneumococcal conjugate composition when the rubber stoppers were coated with either Tween80/WFI or Tween80/ethanol.

TABLE 7
TWENTY-FOUR HOUR STABILITY OF 13VPNC IN THE
PRESENCE OF RUBBERS STOPPERS COATED WITH
TWEEN80/WFI

| | Final [Tween80] | | |
|---|---|---|---|
| | in WFI | | |
| | 0% | | |
| | (control) | 1% | 10% |
| 13vPnC (mL) | 10 | 10 | 10 |
| # of Stoppers | 5 | 5 | 5 |
| Particulates | Yes | No | No |

TABLE 8
TWENTY-FOUR HOUR STABILITY OF 13VPNC IN THE
PRESENCE OF RUBBERS STOPPERS COATED WITH
TWEEN80/ETHANOL

| | Final [Tween80] | | |
|---|---|---|---|
| | in ethanol | | |
| | 0% | | |
| | (control) | 1% | 10% |
| 13vPnC (mL) | 10 | 10 | 10 |
| # of Stoppers | 5 | 5 | 5 |
| Particulates | Yes | No | No |

### REFERENCES

Baldwin, “Contamination of insulin by silicone oil: A potential hazard of plastic insulin syringes”, Diabet. Med., 5:789-790, 1988.
Bernstein, “Clouding and deactivation of clear (regular) human insulin: Association with silicone oil from disposable syringes?”, Diabetes Care, 10:786-787, 1987.
Chantelau and Berger, “Pollution of insulin with silicone oil, a hazard of disposable plastic syringes”, Lancet, 1:1459, 1985.
Chantelau et al., “Silicone oil released from disposable insulin syringes”, Diabetes care, 9:672-673, 1986.
Chantelau, “Silicone oil contamination of insulin”, Diabet. Med., 6:278, 1989.
Collier and Dawson, “Insulin syringes and silicone oil”, Lancet, 5:789-790, 1985.
Jones et al., “Silicone Oil Induced Aggregation of Proteins”, J. Pharmaceutical Sci., 94(4):918-927, 2005.
Bolgiano et al., “Effect of Physico-Chemical Modification on the Immunogenicity of Haemophilus influenzae Type b Oligosaccharide-CRM₁₉₇ Conjugate Vaccines”, Vaccine, 19:3189-3200, 2001.
Ho et al., “Solution Stability of the Subunit Components of Meningococcal C Oligosaccharide-CRM₁₉₇ Conjugate Vaccines”, Biotech. Appl. Biochem., 33:91-98, 2001.
Ho et al., “Physico-Chemical and Immunological Examination of the Thermal Stability of Tetanus Toxoid Conjugate Vaccines”, Vaccine, 20:3509-3522, 2002.

What is claimed is:

1. A process for inhibiting precipitation of a polysaccharide-protein conjugate formulation contained in a container means, the process comprising (a) coating the container means with a water/surfactant solution and (b) adding a polysaccharide-protein conjugate formulation to the coated container means.

2. The process of claim 1, wherein the coated container means in (a) is dried before adding the polysaccharide-protein conjugate formulation of (b).

3. The process of claim 1, wherein the container means is selected from one or more of the group consisting of a vial, a vial stopper, a vial closure, a glass closure, a rubber closure, a plastic closure, a syringe, a syringe stopper, a syringe plunger, a flask, a beaker, a graduated cylinder, a fermentor, a bioreactor, tubing, a pipe, a bag, a jar, an ampoule, a cartridge and a disposable pen.

4. The process of claim 1, wherein the surfactant is polysorbate 80.

5. The process of claim 4, wherein the final concentration of the polysorbate 80 in the water/surfactant solution is at least 0.1% to 10% polysorbate 80 by volume of the water/surfactant solution.

6. The process of claim 1, wherein the polysaccharide-protein conjugate formulation comprises one or more pneumococcal polysaccharides.

7. The process of claim 6, further comprising one or more meningococcal polysaccharides.

8. The process of claim 6, further comprising one or more streptococcal polysaccharides.

9. The process of claim 1, wherein the polysaccharide-protein conjugate formulation is a 7-valent pneumococcal conjugate (7vPnC) formulation comprising a S. pneumoniae serotype 4 polysaccharide conjugated to a CRM₁₉₇ polypeptide, a S. pneumoniae serotype 6B polysaccharide conjugated to a CRM₁₉₇ polypeptide, a S. pneumoniae serotype 9V polysaccharide conjugated to a CRM₁₉₇ polypeptide, a S. pneumoniae serotype 14 polysaccharide conjugated to a CRM₁₉₇ polypeptide, a S. pneumoniae serotype 18C polysaccharide conjugated to a CRM₁₉₇ polypeptide, a S. pneumoniae serotype 19F polysaccharide conjugated to a CRM₁₉₇ polypeptide and a S. pneumoniae serotype 23F polysaccharide conjugated to a CRM₁₉₇ polypeptide.

10. The process of claim 1, wherein the polysaccharide-protein conjugate formulation is a 13-valent pneumococcal conjugate (13vPnC) formulation comprising a S. pneumoniae serotype 4 polysaccharide conjugated to a CRM₁₉₇ polypeptide, a S. pneumoniae serotype 6B polysaccharide conjugated to a CRM₁₉₇ polypeptide, a S. pneumoniae serotype 9V polysaccharide conjugated to a CRM₁₉₇ polypeptide, a S. pneumoniae serotype 14 polysaccharide conjugated to a CRM₁₉₇ polypeptide, a S. pneumoniae serotype 18C polysaccharide conjugated to a CRM₁₉₇ polypeptide, a S. pneumoniae serotype 19F polysaccharide conjugated to a CRM₁₉₇ polypeptide, a S. pneumoniae serotype 23F polysaccharide conjugated to a CRM₁₉₇ polypeptide, a S. pneumoniae serotype 1 polysaccharide conjugated to a CRM₁₉₇ polypeptide, a S. pneumoniae serotype 3 polysaccharide conjugated to a CRM₁₉₇ polypeptide, a S. pneumoniae serotype 5 polysaccharide conjugated to a CRM₁₉₇ polypeptide, a S. pneumoniae serotype 6A polysaccharide conjugated to a CRM₁₉₇ polypeptide, a S. pneumoniae serotype 7F polysaccharide conjugated to a CRM₁₉₇ polypeptide and a S. pneumoniae serotype 19A polysaccharide conjugated to a CRM₁₉₇ polypeptide

11. A process for inhibiting precipitation of a polysaccharide-protein conjugate formulation contained in a container means, the process comprising (a) coating the container means with an ethanol/surfactant solution and (b) adding a polysaccharide-protein conjugate formulation to the coated container means.

12. The process of claim 11, wherein the coated container means in (a) is dried before adding the polysaccharide-protein conjugate formulation of (b).

13. The process of claim 11, wherein the container means is selected from one or more of the group consisting of a vial, a vial stopper, a vial closure, a glass closure, a rubber closure, a plastic closure, a syringe, a syringe stopper, a syringe plunger, a flask, a beaker, a graduated cylinder, a fermentor, a bioreactor, tubing, a pipe, a bag, a jar, an ampoule, a cartridge and a disposable pen.

14. The process of claim 11, wherein the surfactant is polysorbate 80.

15. The process of claim 14, wherein the final concentration of the polysorbate 80 in the ethanol/surfactant solution is at least 0.1% to 10% polysorbate 80 by volume of the ethanol/surfactant solution.

16. The process of claim 11, wherein the polysaccharide-protein conjugate formulation comprises one or more pneumococcal polysaccharides.

17. The process of claim 11, further comprising one or more meningococcal polysaccharides.

18. The process of claim 11, further comprising one or more streptococcal polysaccharides.

19. The process of claim 11, wherein the polysaccharide-protein conjugate formulation is a 7-valent pneumococcal conjugate (7vPnC) formulation comprising a S. pneumoniae serotype 4 polysaccharide conjugated to a CRM₁₉₇ polypeptide, a S. pneumoniae serotype 6B polysaccharide conjugated to a CRM₁₉₇ polypeptide, a S. pneumoniae serotype 9V polysaccharide conjugated to a CRM₁₉₇ polypeptide, a S. pneumoniae serotype 14 polysaccharide conjugated to a CRM₁₉₇ polypeptide, a S. pneumoniae serotype 18C polysaccharide conjugated to a CRM₁₉₇ polypeptide, a S. pneumoniae serotype 19F polysaccharide conjugated to a CRM₁₉₇ polypeptide and a S. pneumoniae serotype 23F polysaccharide conjugated to a CRM₁₉₇ polypeptide.

20. The process of claim 11, wherein the polysaccharide-protein conjugate formulation is a 13-valent pneumococcal conjugate (13vPnC) formulation comprising a S. pneumoniae serotype 4 polysaccharide conjugated to a CRM₁₉₇ polypeptide, a S. pneumoniae serotype 6B polysaccharide conjugated to a CRM₁₉₇ polypeptide, a S. pneumoniae serotype 9V polysaccharide conjugated to a CRM₁₉₇ polypeptide, a S. pneumoniae serotype 14 polysaccharide conjugated to a CRM₁₉₇ polypeptide, a S. pneumoniae serotype 18C polysaccharide conjugated to a CRM₁₉₇ polypeptide, a S. pneumoniae serotype 19F polysaccharide conjugated to a CRM₁₉₇ polypeptide, a S. pneumoniae serotype 23F polysaccharide conjugated to a CRM₁₉₇ polypeptide, a S. pneumoniae serotype 1 polysaccharide conjugated to a CRM₁₉₇ polypeptide, a S. pneumoniae serotype 3 polysaccharide conjugated to a CRM₁₉₇ polypeptide, a S. pneumoniae serotype 5 polysaccharide conjugated to a CRM₁₉₇ polypeptide, a S. pneumoniae serotype 6A polysaccharide conjugated to a CRM₁₉₇ polypeptide, a S. pneumoniae serotype 7F polysaccharide conjugated to a CRM₁₉₇ polypeptide and a S. pneumoniae serotype 19A polysaccharide conjugated to a CRM₁₉₇ polypeptide

21. A process for siliconizing a container means for containing a polysaccharide-protein conjugate formulation, wherein the process inhibits precipitation of the polysaccharide-protein conjugate formulation comprised in the container means, the process comprising (a) coating the container means with a silicone oil/surfactant solution and (b) adding the polysaccharide-protein conjugate formulation to the siliconized container means.

22. The process of claim 21, wherein the container means in (a) is dried before adding the polysaccharide-protein conjugate formulation of (b).

23. The process of claim 21, wherein the coated container means is selected from one or more of the group consisting of a vial, a vial stopper, a vial closure, a glass closure, a rubber closure, a plastic closure, a syringe, a syringe stopper, a syringe plunger, a flask, a beaker, a graduated cylinder, a fermentor, a bioreactor, tubing, a pipe, a bag, a jar, an ampoule, a cartridge and a disposable pen.

24. The process of claim 21, wherein the surfactant is polysorbate 80.

25. The process of claim 24, wherein the final concentration of the polysorbate 80 in the silicone oil/surfactant solution is at least 0.1% to 10% polysorbate 80 by volume of the silicone oil/surfactant solution.

26. The process of claim 21, wherein the polysaccharide-protein conjugate formulation comprises one or more pneumococcal polysaccharides.

27. The process of claim 26, further comprising one or more meningococcal polysaccharides.

28. The process of claim 26, further comprising one or more streptococcal polysaccharides.

29. The process of claim 21, wherein the polysaccharide-protein conjugate formulation is a 7-valent pneumococcal conjugate (7vPnC) formulation comprising a S. pneumoniae serotype 4 polysaccharide conjugated to a CRM₁₉₇ polypeptide, a S. pneumoniae serotype 6B polysaccharide conjugated to a CRM₁₉₇ polypeptide, a S. pneumoniae serotype 9V polysaccharide conjugated to a CRM₁₉₇ polypeptide, a S. pneumoniae serotype 14 polysaccharide conjugated to a CRM₁₉₇ polypeptide, a S. pneumoniae serotype 18C polysaccharide conjugated to a CRM₁₉₇ polypeptide, a S. pneumoniae serotype 19F polysaccharide conjugated to a CRM₁₉₇ polypeptide and a S. pneumoniae serotype 23F polysaccharide conjugated to a CRM₁₉₇ polypeptide.

30. The process of claim 21, wherein the polysaccharide-protein conjugate formulation is a 13-valent pneumococcal conjugate (13vPnC) formulation comprising a S. pneumoniae serotype 4 polysaccharide conjugated to a CRM₁₉₇ polypeptide, a S. pneumoniae serotype 6B polysaccharide conjugated to a CRM₁₉₇ polypeptide, a S. pneumoniae serotype 9V polysaccharide conjugated to a CRM₁₉₇ polypeptide, a S. pneumoniae serotype 14 polysaccharide conjugated to a CRM₁₉₇ polypeptide, a S. pneumoniae serotype 18C polysaccharide conjugated to a CRM₁₉₇ polypeptide, a S. pneumoniae serotype 19F polysaccharide conjugated to a CRM₁₉₇ polypeptide, a S. pneumoniae serotype 23F polysaccharide conjugated to a CRM₁₉₇ polypeptide, a S. pneumoniae serotype 1 polysaccharide conjugated to a CRM₁₉₇ polypeptide, a S. pneumoniae serotype 3 polysaccharide conjugated to a CRM₁₉₇ polypeptide, a S. pneumoniae serotype 5 polysaccharide conjugated to a CRM₁₉₇ polypeptide, a S. pneumoniae serotype 6A polysaccharide conjugated to a CRM₁₉₇ polypeptide, a S. pneumoniae serotype 7F polysaccharide conjugated to a CRM₁₉₇ polypeptide and a S. pneumoniae serotype 19A polysaccharide conjugated to a CRM₁₉₇ polypeptide

31. A polysaccharide-protein conjugate formulation comprised in a container means prepared according to the process of claim 1.

32. A polysaccharide-protein conjugate formulation comprised in a container means prepared according to the process of claim 11.

33. A polysaccharide-protein conjugate formulation comprised in a container means prepared according to the process of claim 21.
